(19) United States
(12) Patent Application Publication
(10) Pub. No.: US 2003/0045797 A1
(43) Pub. Date: Mar. 6, 2003

(54) Automatic optimization of doppler display parameters

(76) Inventors: Donald Christopher, Everett, WA (US); Marshall Robinson, Snohomish, WA (US); Helen Routh, Seattle, WA (US); Claudio Simon, Seattle, WA (US); Ahmed Morsy, Bellevue, WA (US); Keith Johnson, Lynnwood, WA (US); Patrick Rene Pesque, Scottsdale, AZ (US)

Correspondence Address: W. Brinto Yorks, Jr., ATL Ultrasound, Inc., 22100 Bothell Everett Highway, P.O. Box 3003, Bothell, WA, 98041-3003, US

(21) Appl. No.: 09/941,348

(22) Filed: Aug. 28, 2001

(51) Int. Cl. A61B 8/00; A61B 8/12; A61B 8/14; A61B 8/02; A61B 8/06

(52) U.S. Cl. 600/453

(57) ABSTRACT

In an ultrasonic diagnostic imaging system, the parameters which govern the display of Doppler information are automatically optimized to make better use of the display range or area. Spectral Doppler information may be used to optimize a spectral display or a colorflow display, and colorflow Doppler information may be used to optimize a spectral display or a colorflow display. The optimization may be invoked by a manual user control which automatically optimizes one or a plurality of display parameters. Automatic optimization may be invoked only when called for by the user, or periodically after a time interval, a given number of heart cycles, or when the user has made a change to the display or imaging mode. Preferably the optimization processor runs continuously in the background so that optimized parameters are available immediately when called for. The optimization processor may utilize “hidden” Doppler data which has been acquired but is not used for display purposes.

This invention relates to ultrasonic diagnostic imaging systems and, in particular, to ultrasonic diagnostic imaging systems in which Doppler display parameters are automatically optimized.

Doppler imaging is performed when a clinician desires to acquire information about the flow of blood or moving tissues of a patient. The display of flow or motion velocity may be done by means of a spectral Doppler display in which velocities are displayed graphically, or by a color Doppler display in which velocities are displayed in shades or hues of color. In both cases the range of velocities displayed is bounded by graphical or color limits set by the continuous wave (cw) Doppler sampling rate, or the pulsed wave (pw) pulse repetition frequency (PRF). In many cases the range of blood or tissue velocities cannot be accurately predicted before the exam begins, and hence the clinician must make a number of adjustments as the exam commences and progresses in order to maximize the range of Doppler frequencies in the display and the resolution of the different velocities, and to minimize aliasing. Generally the clinician must adjust two or three controls in order to obtain the optimal display in the system's display area. It would be desirable to automate this adjustment process so that an optimal display is produced with little or no need for manual adjustment, enabling the clinician to gather optimized data upon commencement and progression of the exam.

In accordance with the principles of the present invention an ultrasonic diagnostic imaging system is provided in which Doppler settings such as the Doppler PRF and the display baseline (position and polarity) are automatically optimized by the ultrasound system. The clinician can decide whether to have one, several, or all of the Doppler display parameters optimized automatically, and the periodicity with which optimization is updated. The spectral Doppler PRF and baseline offset and inversion can be automatically optimized using data within the spectral trace or color data of a corresponding color M-mode trace or color Doppler image. The color Doppler image PRF and baseline can be automatically optimized using its own color Doppler estimation data or data within a corresponding spectral Doppler trace or color M-mode display. The color M-mode PRF and baseline can be automatically optimized using its own Doppler estimation data or data within a corresponding spectral Doppler trace or color Doppler display. This optimization can be performed on either live, real time displays or on displays of stored data such as Doppler Cineloop® information. The optimization calculations can be made using only displayed data, or data which is acquired and “hidden” from the user. These optimization techniques can be applied to all relevant Doppler targets such as blood flow, moving tissue, and contrast agents, and can be applied in all color Doppler modes such as velocity colorflow, color power imaging, tissue Doppler imaging, and power motion imaging, and in all spectral Doppler modes such as continuous wave, pulse wave, single angle, and vector Doppler.

In the drawings:

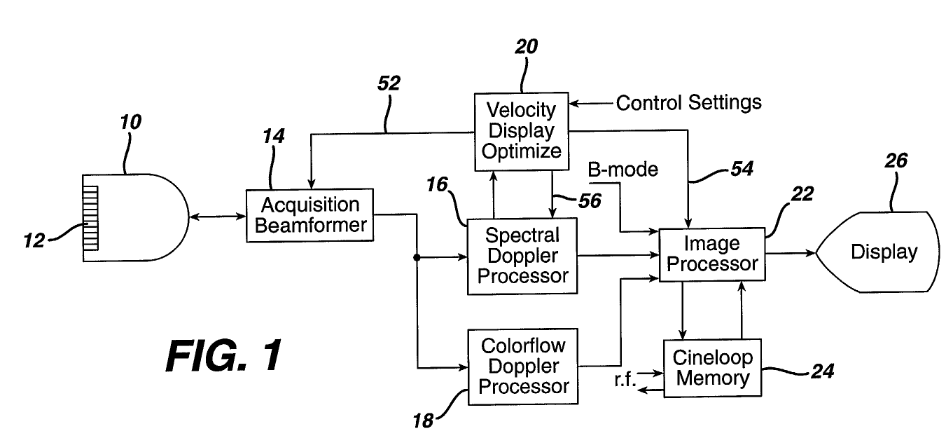

FIG. 1 illustrates in block diagram form an ultrasound system constructed in accordance with the principles of the present invention in which spectral Doppler data is used to automatically optimize a spectral Doppler display;

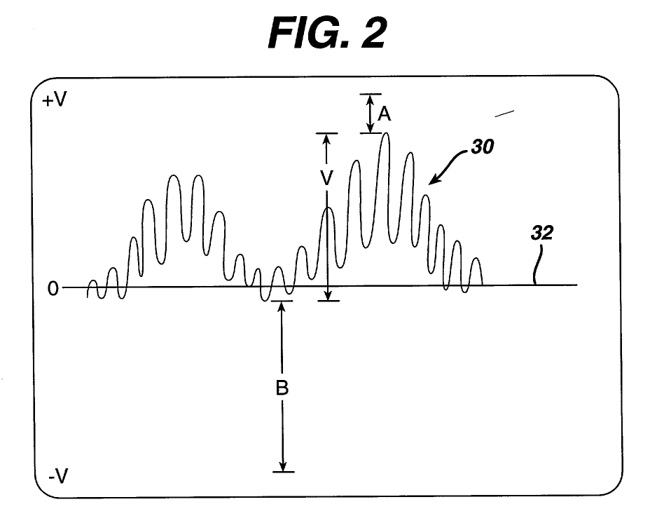

FIG. 2 illustrates spectral Doppler variables used to optimize a Doppler display;

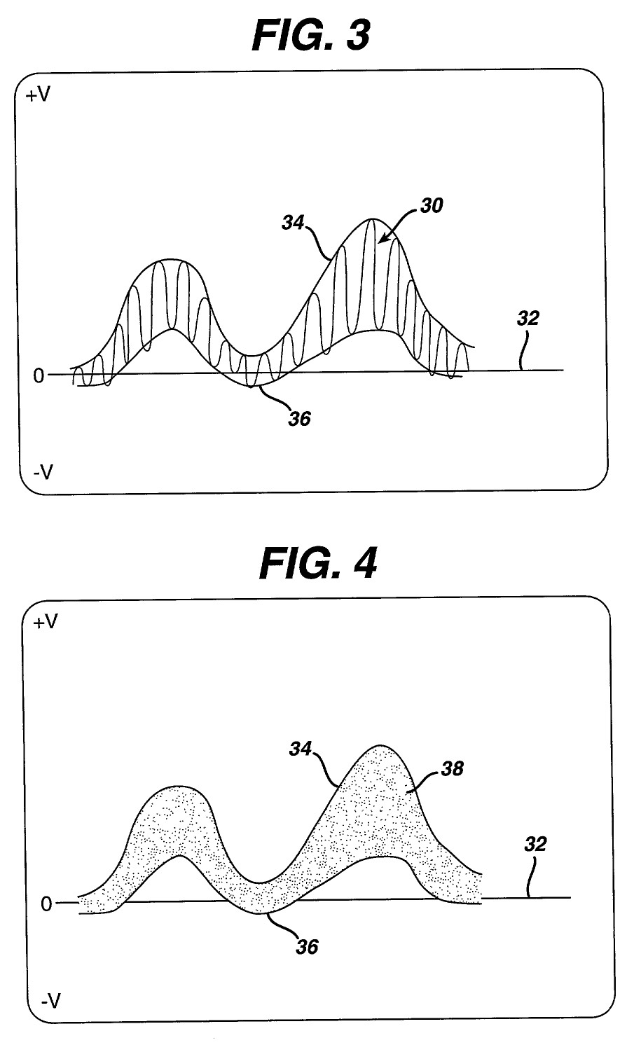

FIGS. 3 and 4 illustrate different ways to delineate spectral Doppler data for display optimization;

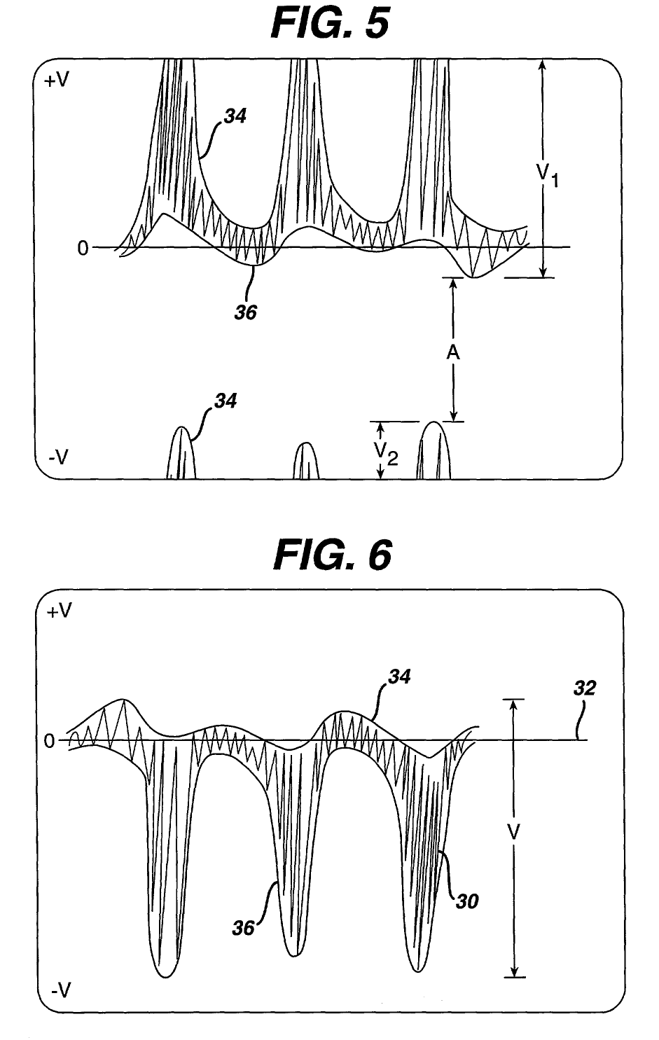

FIG. 5 illustrates the detection and reduction of aliasing in accordance with the present invention;

FIG. 6 illustrates the automatic inversion of a Doppler waveform;

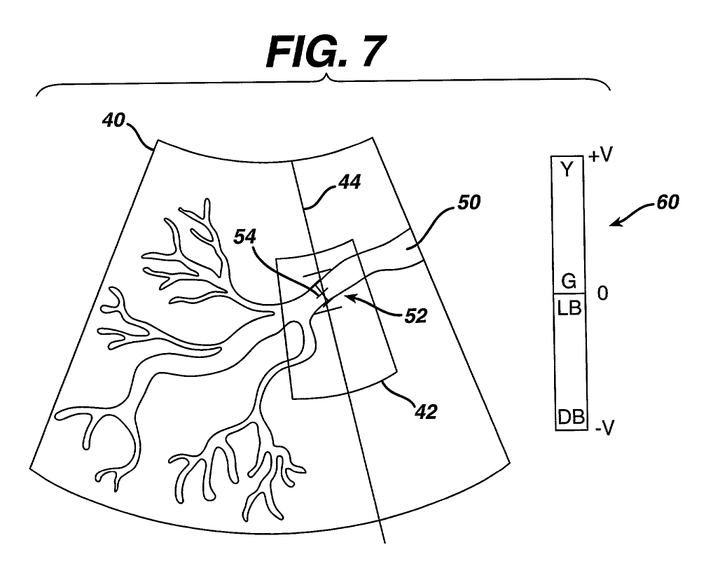

FIGS. 7 and 9 illustrate the use of colorflow data to optimize a spectral Doppler display;

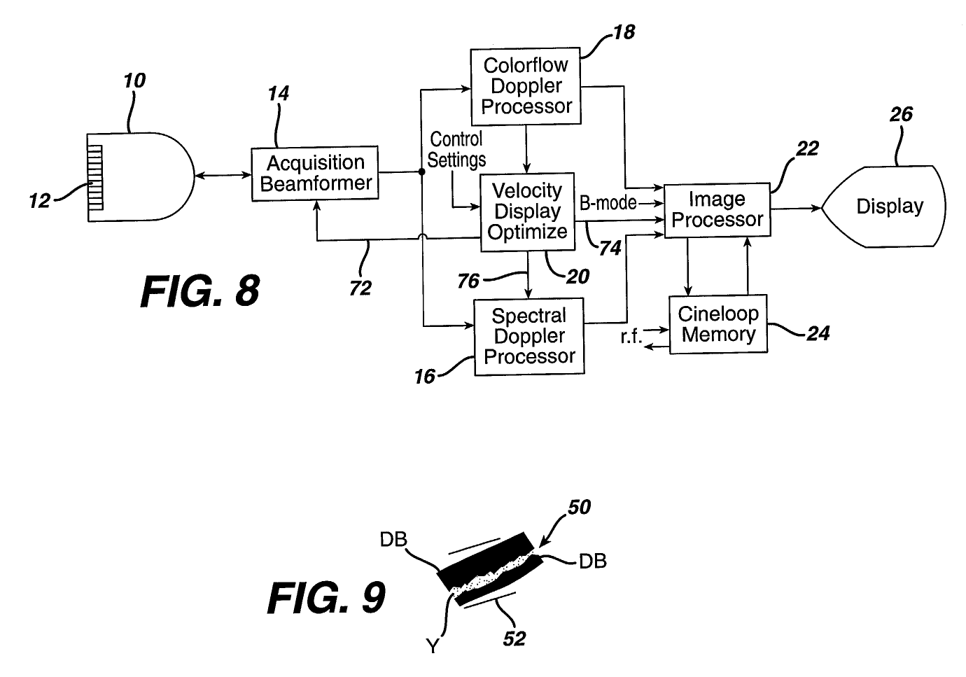

FIG. 8 illustrates in block diagram form another embodiment of the present invention in which colorflow data is used to optimize a spectral Doppler display;

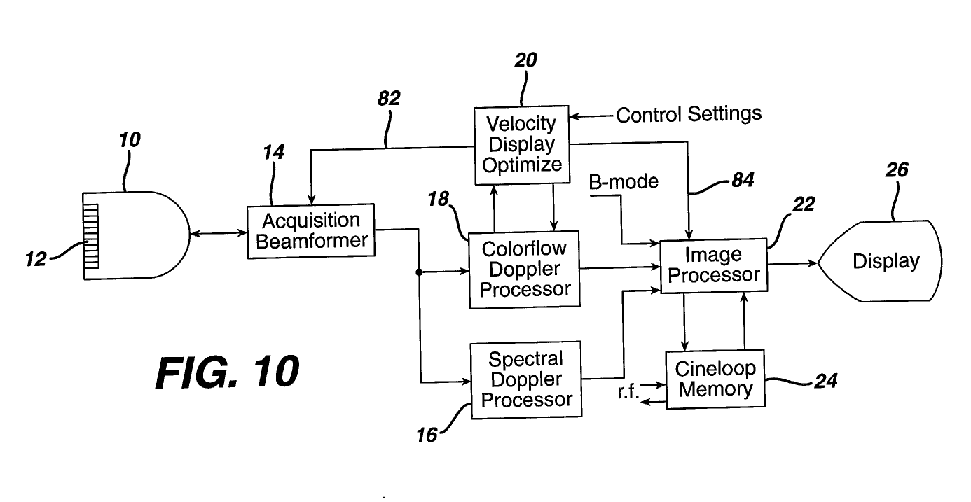

FIG. 10 illustrates in block diagram form another embodiment of the present invention in which colorflow data is used to optimize a colorflow Doppler display;

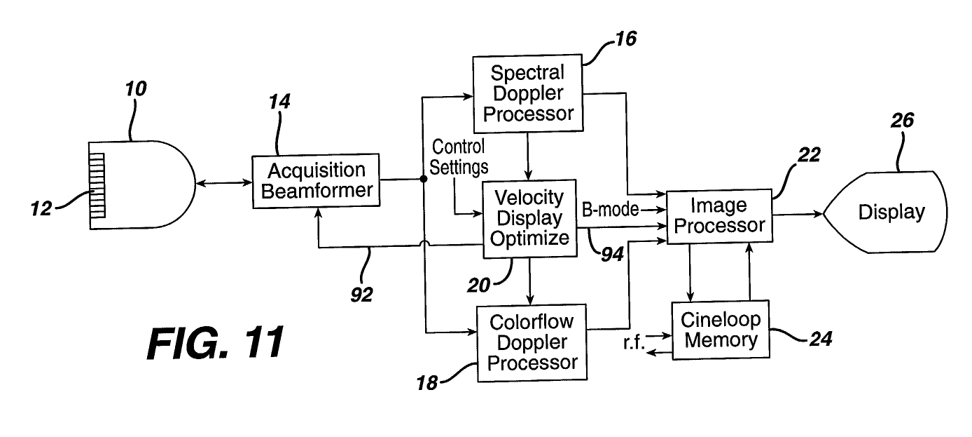

FIG. 11 illustrates in block diagram form another embodiment of the present invention in which spectral Doppler data is used to optimize a colorflow Doppler display; and

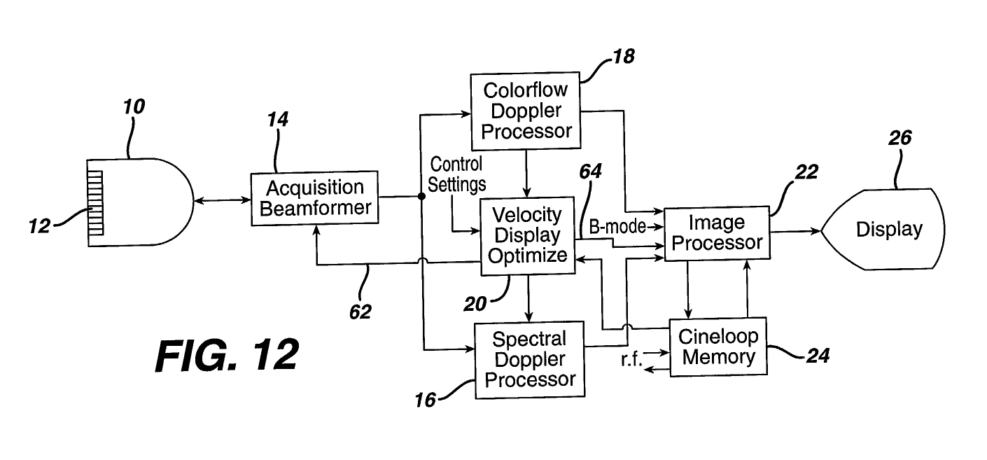

FIG. 12 illustrates in block diagram form another embodiment of the present invention in which Doppler data stored in Cineloop memory is optimized in accordance with the principles of the present invention.

Referring first to FIG. 1, an ultrasound system constructed in accordance with the principles of the present invention is shown in block diagram form. In this embodiment spectral data is used to automatically optimize the pw PRF, baseline position, or baseline inversion of a spectral Doppler display. A scanhead 10 having an ultrasonic transducer 12 transmit ultrasonic waves and receives ultrasonic echo signals. The received echo signals may be at the same frequency as the transmit frequency, or at a higher or lower harmonic of the transmit frequency. Control of the transducer transmission and processing of the received echo signals is provided by an acquisition beamformer 14. The coherent echo signals may be detected and processed for B mode display, may be coupled to Doppler processors 16 and 18 for spectral and/or colorflow display, or may be used for both B mode and Doppler display as described in U.S. Pat. No. 6,139,501. The processed B mode and Doppler signals are coupled to an image processor 22 where they are processed for display in the desired image format and are then displayed on an image display 26. Sequences of real time images may be captured and stored in a Cineloop memory 24 in r.f., estimate, native, or composite display form, from which they may be replayed for more detailed analysis or reprocessed as described below.

In accordance with the principles of the present invention a velocity display optimizer 20 analyzes spectral Doppler data and uses the results of the analysis to automatically adjust parameters of a spectral Doppler display such as the velocity range (PRF), Doppler baseline position, and baseline inversion. In the illustrated embodiment the velocity display optimizer receives spectral data from the spectral Doppler processor 16 and returns display parameters for the spectral Doppler display. The velocity display optimizer sends control parameters such as those for the PRF, sample volume size and tracking, and the transmit steering and D-line position over line 52 to the acquisition portion of the ultrasound system. Wall filter, sample volume depth, and scroll speed are among the parameters which are supplied to the spectral Doppler processor over line 56. Parameters such as the baseline positioning, inversion, color map, Doppler angle correction, and sample volume tracking are among those supplied to the image processor over line 54. In a preferred embodiment the velocity display optimizer 20 adjusts those parameters denominated by control setting set by the user. For example, the user may have individual hard or softkeys available to turn the automatic adjustment of particular parameters “on” or “off.” The user may select from an “Auto PRF” button, an “Auto Baseline” button, and/or an “Auto Invert” button, for instance. Setting one of these controls conditions the ultrasound system to set the particular parameter automatically. The user may also be able to select an “Auto Doppler” button to invoke automatic adjustment for all of the Doppler parameters. The automatic adjustment may occur periodically with the passage of time or in response to an operational event such as a mode change, and ECG trigger signal, or after a predetermined heart rate interval. Leaving the button “off” requires the user to set the Doppler parameters manually in the conventional manner.

A spectral Doppler display is shown in FIG. 2. This drawing illustrates a spectral waveform 30 in reference to a zero velocity baseline 32. The baseline is centered between two velocity limits +V and −V, the maximum velocities in opposite directions which are represented without aliasing. The velocity limits are set directly by the cw Doppler sampling rate or the pw PRF, with the two limits being equal to the Nyquist limits of the sampling rate, +PRF/2 and −PRF/2. Conventionally the user will set the PRF and the display will use the PRF setting to establish the +V and −V limits of the display with the baseline centered between the two as shown in FIG. 2. But since the Doppler waveform cannot be predicted in advance, the spectral waveform can appear predominately in one portion of the display. In FIG. 2 the spectral waveform 30 is seen to be principally in the upper half of the display since it is predominately above the baseline and the lower half of the display is unused. The user can generally manually adjust the display parameters to make better use of the display area, but it is desirable for the ultrasound system to do this automatically as the present invention provides. The velocity display optimizer 20 knows the PRF and uses the spectral data to measure the values A and B shown in the drawing, where A is the range between the peak positive spectral excursion and the upper display limit and B is the range between the peak negative spectral excursion and the lower display limit. The velocity display optimizer calculates new ranges A′ and B′, where

A′≅B′≅(A+B)/2

The velocity display optimizer provides these A′ and B′ values to the spectral Doppler processor 16 or the image processor 22 and the spectral display is remapped to a display which uses these values, which centers spectral waveform 30 in the center of the display. This centering of the waveform repositions the baseline 32 by an increment (B-B′) and resets the +V and −V display limit values, which may no longer be of equal magnitudes in the display for an asymmetrical waveform such as waveform 30. If desired, the A′ and B′ values can be reduced so that the spectral display is remapped to an enlarged display which makes better use of the full display height. The spectral waveform can be displayed over the full height of the display, but preferably a guard range A″ and B″ is left to allow for subsequent peak excursions which exceed the previous maximum positive and negative excursions. Preferably, rather than simply enlarging the spectral waveform by remapping, the velocity display optimizer calculates a new PRF value from the Doppler equation such as

PRF′∝V+(A′+B′)

where V is the velocity range between the peak positive and negative excursions of the spectral waveform as shown in the drawings and A′ and B′ (or A″ and B″) are determined as described above. If the excursion range V of the spectral waveform is too small, the PRF is reduced to make better use of the display window. The new PRF′ value is applied to the acquisition beamformer 14 as shown in FIG. 1, causing the motion or flow to be sampled at a more effective sampling rate for display. As a result of the foregoing adjustments, the spectral display is rescaled by the PRF change and the spectral waveform is translated to be remapped more effectively to the spectral display area.

This adjustment process may take advantage of other processing of the spectral waveform. For instance, U.S. Pat. Nos. 5,287,753 and 5,634,465 describe techniques for tracing the mean and the peak of a spectral Doppler waveform. These analytical techniques may be invoked by the user, in which case the velocity display optimizer 20 can readily obtain the peak maximum and minimum excursions of the spectral waveform directly from the traces 34 and 36 of the peak positive and negative excursions of the waveform, as illustrated in FIG. 3. If the user has not invoked an automatic tracing option, the waveform tracing may be done by the velocity display optimizer without display of the traces 34 or 36 to the user, and the peak maximum and minimum excursions taken from the undisplayed (“hidden”) traces.

Another analytical approach to determine the adjustments to be made is to recognize that for these calculations the actual spectral data is not needed; rather, it is the locus of the spectral data in the display space which is of importance. Thus, the spectral data can be remapped to a binary representation, where the location of valid spectral data is encoded as a “1”, and other display areas encoded as a “0”. The adjustments are then calculated from such a binary map. A binary map can also be produced from the waveform traces 34 and 36 as shown in FIG. 4. In this drawing the area 38 between the waveforms 34 and 36 is encoded as a “1” and the remaining display area is encoded as a “0”. The display parameters are then calculated to maximize the use of the display area by the area 38 between the traces.

The image processing techniques of the present invention may also be used to address aliasing problems. In FIG. 5 the motion is undersampled by the PRF, resulting in aliasing. Instead of having two unused areas A and B and a single peak-to-peak region V, the display has two waveform areas delineated by V₁ and V₂ at the top and bottom of the display, separated by a single unused area A. The V₁ and V₂ excursions extend fully to the +V and −V Nyquist limits. This condition is readily recognized and the velocity display optimizer responds by increasing the PRF of the acquisition beamformer 14. Preferably the baseline would be adjusted before adjusting the PRF. Incremental changes in the PRF may be invoked until the aliasing condition is no longer present.

One manifestation of a spectral display is that in which large negative excursions predominate, due to the polarity sensing of the Doppler processor as shown in FIG. 6. In such a situation many clinicians want to invert the waveform 30 to its more familiar orientation, which is done by changing the polarity of the Doppler display signals. This condition is readily recognized when the peak-to-peak excursion range V of the spectral waveform 30 is predominately below the baseline 32. When a significant percentage of the excursion V is below the baseline, the velocity display optimizer 20 responds by changing the polarity of the Doppler display signals, thereby inverting the displayed waveform, an adjustment often referred to as “baseline invert.”

Colorflow data may also be used to automatically adjust the parameters of a corresponding spectral Doppler display as shown in FIGS. 7 and 8. FIG. 7 shows a colorflow Doppler image 40 which is used to image the blood flow velocities of the portion of a vessel 50 which is inside a color box 42. A spectral analysis such as that of FIG. 2 is initiated by positioning a sample volume 52 over the center of the blood vessel 50. A flow direction cursor 54 is set to be aligned with the direction of blood flow for angle correction. Preferably the flow direction cursor setting and angle correction is performed automatically as described in U.S. patent [application Ser. No. 09/721,301, filed Nov. 21, 2000]. Next to the colorflow image 40 on the display screen is a color bar 60, which depicts the mapping of the flow colors to a range of velocity values. In this illustration positive velocities extend from green (G) to yellow (Y) in color and negative velocities extend from light blue (LB) to dark blue (DB), where the zero velocity point between green and yellow is the color baseline.

The colorflow data of the image 40 is analyzed by the embodiment of the present invention shown in FIG. 8. In this embodiment the velocity display optimizer 20 receives colorflow data from the colorflow Doppler processor 18 and analyzes this data to automatically adjust the parameters of a spectral Doppler display. Parameters for the PRF, sample volume size, transmit angle, D-line position, and sample volume tracking are among those which are coupled to the acquisition portion of the ultrasound system over line 72. Baseline shift, invert, grayscale mapping and angle correction parameters are among those conducted over line 74. Wall filter and sample volume depth are among those parameters conducted over line 76. The velocity display optimizer 20 looks at the range of values of the color pixels in the colorflow display or, preferably, the range of color pixel in an area around a sample volume selected by the user. The color value at the center of the vessel 50, as delineated by the cursor 54, is picked as a peak velocity value. If the color values occupy a narrow range in comparison with the range of color values used in the color bar 60, e.g., the pixels are virtually all the same color, the PRF is too high and the velocity display optimizer responds by sending a lower PRF setting to the acquisition beamformer 14. The velocity display optimizer also analyzes the color differences of adjacent pixels. If aliasing is present, which in the example of FIG. 7 would be a sudden transition of adjacent pixels from dark blue to yellow as represented in FIG. 9, the PRF is increased by the velocity display optimizer. Alternatively, a baseline shift can be used to cure aliasing. The A and B values are computed from the differences between the limits of the color range of the colorflow pixels in the image 40 and the +V and −V values of the color bar 60, and are used to shift the spectral baseline and make the most effective use of the spectral display area as described above. Thus, the spectral display parameters have been optimized using colorflow data.

In the embodiment of the present invention shown in FIG. 10, colorflow data is used to optimize the colorflow display. The velocity display optimizer 20 receives the color pixel values from the colorflow Doppler processor 18. Adjacent pixels are compared for sudden color transitions from the +V color to the −V color of the color bar 60, in which case the PRF parameter on line 82 is changed by the velocity display optimizer to increase the PRF and reduce aliasing. The range of color values of the pixels is analyzed and, if it is too small, the PRF is reduced. Peak color values are detected to detect the ranges between them and the +V and −V color bar limits and used to make the A, B, and V determinations described above. These values are used to remap the PRF limits, color baseline, and range of pixel colors to obtain the best color range for the colorflow image 40. Parameters for the baseline and color map are applied to the image processor 22 over line 84.

FIG. 11 illustrates a further embodiment of the present invention in which spectral data is used to optimize the parameters of a colorflow display. The velocity display optimizer obtains spectral data from the spectral Doppler processor 16 and determines the maximum and minimum excursions of the spectral waveform, preferably from automatically computed tracings as described above. From these excursion values and the +V and −V limits of the spectral display the A, B, and V values are computed as described above. These values are then used as described above to reset the PRF if necessary by way of line 92 to reduce aliasing or expand the waveform and hence the range of colors for the colorflow display. A′ and B′ are computed to locate the colorflow baseline (zero value) for the color bar, and a display range of colors is mapped and parameters for the baseline and color map applied to the image processor 22 over line 94. This processing may occur after the colorflow image has been acquired, and it may also take place using a B mode image with the sample volume for the spectral data positioned over a blood vessel. The foregoing adjustments are computed and then the color display commences over the B mode image using the optimized values for display.

FIG. 12 illustrates a further embodiment of the present invention where adjustment of Doppler display parameters is performed on data stored in Cineloop memory. In a constructed embodiment the user pushes the “Freeze” button to stop real time acquisition and retain the most recently acquired images in the Cineloop memory 24. The number of images saved in the sequence or “loop” is dependent upon the size of the Cineloop memory, which may retain in excess of 100 frames. The images stored in the memory 24 are coupled to the velocity display optimizer 20 where the Doppler data of the images is used to optimize the display parameters of the colorflow or spectral images stored in Cineloop, or both using one or more of the optimization techniques described above. The image data and the new display parameters for the baseline, display inversion, and/or color map are then applied to the image processor 22 over line 64 where the images are displayed in accordance with the new display scaling or mapping. Automatic optimization can be invoked after the Freeze button has been depressed to save the images. The optimization can be based upon the Doppler data of an image shown on the display screen, on the Doppler data of a defined region of interest of the images of the loop, or using all the Doppler data of the whole loop. The latter two implementations will optimize all of the loop images by, for instance, preventing aliasing in all the images of the loop. The images of the Cineloop can also be acquired at the system's maximum PRF, then optimized to scale the Doppler data to the display area for the optimal presentation of alias-free images.

There are various ways in which the automatic optimization can be invoked. One approach is to optimize the images only when the user selects the Auto Optimize button based upon the Doppler data of one or a few heart cycles, such as the data of the heart cycles present shown in a spectral display. Automatic optimization can be invoked periodically every few heart cycles or every few seconds to maintain optimization. Initially automatic optimization may be deferred if an insufficient number of heart cycles have been acquired until a sufficient amount of data has been acquired and is available for the optimization calculations. Optimization can be automatically invoked each time the user moves the sample volume or each time the color box 42 is adjusted or reset. Optimization can be automatically invoked each time the imaging mode is changed, for instance, when changing from B mode to color or when starting spectral data acquisition. Preferably, the velocity display optimizer runs continuous in the background to produce optimized parameters during Doppler acquisition even when automatic optimization has not been invoked by the user. By doing so, optimized parameters are immediately available whenever the user chooses to automatically optimize a Doppler display.

When automatic optimization is invoked for a scrolling display such as a spectral Doppler or color M-mode display, it is preferable not to simply apply the optimized parameters to subsequently acquired Doppler information. Rather, it is preferred that the optimized parameters be applied to all of the Doppler information on the screen, so that the user sees all of the Doppler data in the display area displayed with the optimized parameters.

Other acquisition or display parameters may also be automatically optimized in accordance with the principles of the present invention. For instance, when strong contrast agent signals appear, the signal gain may be automatically adjusted to reduce blooming in the image due to excessive contrast signal saturation. The dynamic range, noise floor, color box, transmit angle, and audio volume are other parameters which may be adjusted automatically. The techniques of the present invention may be employed to optimize the display of both 2D and 3D ultrasound images, and of 1D images such as color M-mode displays.

It will be appreciated that a given embodiment of the present invention need not optimize all of the display parameters as described above, but may leave some parameters for only manual adjustment. For instance, automatic remapping to a display area or range of display colors may be invoked automatically, with the PRF continuing to be adjustable only by user control.

What is claimed is:

1. A method for optimizing the display of Doppler ultrasound information comprising:
receiving Doppler signal information;
processing Doppler signal information for display in a display area; and
analyzing spectral Doppler signal information to produce optimized display parameters for display of the processed Doppler signal information in the display area.

2. The method of claim 1, wherein processing further comprises processing Doppler signal information for spectral display.

3. The method of claim 2, wherein analyzing further comprises analyzing spectral Doppler signal information to optimize at least one of the parameters of the PRF, the spectral Doppler display area, the display polarity, and the Doppler baseline position.

4. The method of claim 3, wherein the optimized display parameters map the processed Doppler signal information to make more extensive use of the display area.

5. The method of claim 1, wherein processing further comprises processing Doppler signal information for an anatomical Doppler display.

6. The method of claim 5, wherein processing further comprises processing Doppler signal information for display of a colorflow Doppler display in the display area.

7. The method of claim 5, wherein processing further comprises processing Doppler signal information for display of a velocity Doppler display in the display area.

8. The method of claim 5, wherein processing further comprises processing Doppler signal information for display of a Doppler M-mode display in the display area.

9. The method of claim 5, wherein processing further comprises processing Doppler signal information for display of a power Doppler display in the display area.

10. The method of claim 6, 7, 8, or 9, wherein analyzing further comprises analyzing spectral Doppler signal information to optimize at least one of the parameters of the PRF, the range of color pixel values, the color baseline position, and the color range polarity.

11. The method of claim 10, wherein the optimized display parameters map the processed Doppler signal information to make more extensive use of the range of color or intensity of displayed Doppler information.

12. A method for optimizing the display of Doppler ultrasound information comprising:
receiving Doppler signal information;
processing Doppler signal information for display of an anatomical Doppler display in a display area; and
analyzing Doppler signal information to produce optimized display parameters for display of the processed Doppler signal information in the display area.

13. The method of claim 12, wherein analyzing further comprises analyzing colorflow Doppler signal information.

14. The method of claim 12, wherein analyzing further comprises analyzing spectral Doppler signal information.

15. The method of claim 12, wherein analyzing further comprises analyzing power Doppler signal information.

16. The method of claim 12, wherein analyzing further comprises analyzing Doppler M-mode signal information.

17. The method of claim 13, 14, 15 or 16, wherein analyzing further comprises analyzing Doppler signal information to optimize at least one of the parameters of the PRF, the range of color pixel values, the color baseline position, and the color range polarity.

18. The method of claim 17, wherein processing further comprises processing Doppler signal information for display of a colorflow Doppler display in the display area.

19. The method of claim 17, wherein processing further comprises processing Doppler signal information for display of a power Doppler display in the display area.

20. The method of claim 17, wherein processing further comprises processing Doppler signal information for display of a Doppler M-mode display in the display area.

21. The method of claim 17, wherein the optimized display parameters act to reduce aliasing in the displayed image.

22. The method of claim 1 or 12, further comprising:
manually actuating a control to cause automatically optimized display parameters to be used for a Doppler display.

23. The method of claim 22, wherein manually actuating a control comprises automatically optimizing a single parameter.

24. The method of claim 22, wherein manually actuating a control comprises automatically optimizing a plurality of parameters.

25. The method of claim 1 or 12, wherein analyzing Doppler signal information to produce optimized display parameters occurs substantially continuously during display of the Doppler display being optimized.

26. The method of claim 1 or 12, further comprising updating the display parameters periodically after one or more heart cycles.

27. The method of claim 1 or 12, wherein analyzing Doppler signal information to produce optimized display parameters occurs periodically after a predetermined time interval during display of a display using optimized parameters.

28. The method of claim 1 or 12, wherein analyzing Doppler signal information to produce optimized display parameters occurs in response to modification of a Doppler setting by the user.

29. The method of claim 1 or 12, further comprising:
storing a sequence of processed Doppler images in memory; and
wherein analyzing comprises analyzing Doppler signal information to produce optimized display parameters for display of the stored Doppler images.

30. The method of claim 29, wherein analyzing comprises analyzing Doppler signal information of a plurality of the images stored in memory.

31. The method of claim 1 or 12, wherein the Doppler signal information which is analyzed comprises a trace of peak spectral Doppler information.

32. The method of claim 1 or 12, wherein the Doppler signal information which is analyzed comprises a binary map delineating Doppler and non-Doppler information.

33. A method for optimizing the display of Doppler ultrasound information comprising:
receiving Doppler signal information;
processing Doppler signal information for display in a display area; and
analyzing Doppler signal information to produce optimized display parameters for display of the processed Doppler signal information in the display area,
wherein analyzing further comprises analyzing at least some Doppler signal information which is not used to produce a displayed image.

34. A method for optimizing the display of Doppler ultrasound information comprising:
receiving Doppler signal information;
processing Doppler signal information for display in a display area; and
analyzing Doppler signal information to produce optimized display parameters for display of the processed Doppler signal information in the display area,
wherein analyzing Doppler signal information occurs substantially continuously during display of the Doppler display being optimized.

35. The method of claim 34, wherein analyzing further comprises analyzing Doppler signal information to produce optimized display parameters prior to the display of the Doppler display being optimized.

36. The method of claim 35, wherein analyzing comprises analyzing Doppler signal information in the background to produce optimized display parameters.

37. The method of claim 35, further comprising displaying a Doppler image which utilizes a plurality of different optimized display parameters as compared to the previously displayed Doppler image.
